SEQUENCE LISTING (1 sequence)

SEQ ID NO: 1 - DNA, 15 bp, Artificial
  c-raf antisense oligonucleotide
gtgctccattgatgc

(19) United States
(12) Patent Application Publication
(10) Pub. No.: US 2006/0165744 A1
(43) Pub. Date: Jul. 27, 2006

(54) Combination liposomal formulations

(75) Inventors: Haris Jamil, Libertyville, IL (US); Imran Ahmad, Wadsworth, IL (US); Zafeer Ahmad, Gurnee, IL (US); Gopal Anyarambhatla, Decatur, IL (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(73) Assignee: NeoPharm, Inc, Lake Forest, IL (US)

(21) Appl. No.: 10/558,159

(22) PCT Filed: May 22, 2004
(86) PCT No.: PCT/US04/16413

§ 371 Date: Feb. 8, 2006

Related U.S. Application Data
(60) Provisional application No. 60/472,664, filed on May 22, 2003.
(60) Provisional application No. 60/495,260, filed on Aug. 13, 2003.

(51) Int. Cl. A01N 25/00; A61K 9/127; C12N 15/88

(52) U.S. Cl. 424/405; 424/450; 435/458

(57) ABSTRACT

The present invention provides a composition comprising a physiologically acceptable carrier and two or more agents encapsulated in a liposome, wherein the combination of the two or more agents possess the following properties: (1) cytotoxicity to tumor cells, (2) nutritional properties, (3) use in application to nails, hair, skin or lips or (4) activity against parasites and insects. The invention also provides a method of making such a composition. The invention further provides a method of treating cancer when the combination of the two or more agents is cytotoxic to tumor cells.

### CROSS REFERENCE TO RELATED APPLICATIONS

This application claims priority to U.S. Provisional Patent Application 60/472,664, filed May 22, 2003. This application also claims priority to U.S. Provisional Patent Application 60/495,260, filed Aug. 13, 2003. The disclosures of these two applications are incorporated in their entireties herein by reference thereto.

### FIELD OF THE INVENTION

This invention pertains to a composition comprising two or more agents (e.g., drugs or other active agents) encapsulated into a liposome.

### BACKGROUND OF THE INVENTION

The treatment of cancer has progressed significantly with the development of drugs that more efficiently target and kill cancer cells. Many cancer types, however, manifest as multifactorial diseases, which often require a multimodal therapeutic approach. In this respect, clinicians have realized limited success with the administration of a single drug to treat a particular type of cancer. Indeed, both preclinical and clinical studies have revealed that chemotherapy regimens employing two or more anticancer drugs produce a synergistic effect on therapeutic efficacy as compared to administration of each drug individually (see, e.g., Damon et al., Cancer Invest., 4, 421-44 (1986), Caponigro et al., Anticancer Drugs, 12, 489-97 (2001), and U.S. Pat. No. 6,469,058). As a result, current chemotherapy protocols typically involve concurrent administration of two or more anticancer drugs to a patient (“combination chemotherapy”).

Although combination chemotherapy has proven to be more effective in killing cancer cells than pharmaceuticals containing only one active agent, each type of therapy has inherent limitations. Many anticancer drugs exhibit an extremely low solubility in water, making it difficult to prepare aqueous formulations of a particular drug. Moreover, repeated administrations of an anticancer drug can produce multidrug resistance in the treated patient, thereby reducing drug efficacy over time. The dose-limiting toxicity of certain drugs also limits their therapeutic potential. Thus, formulations suitable for combination chemotherapy are needed that can overcome the solubility problems associated with anticancer drugs, reduce their toxicity, and enhance their efficacy.

### BRIEF SUMMARY OF THE INVENTION

The invention provides a composition comprising a physiologically acceptable carrier and two or more agents (e.g., drugs or other active agents) encapsulated into a liposome, wherein the combination of the two or more agents possess the following properties: (1) cytotoxicity to tumor cells, (2) nutritional properties, (3) use in application to nails, hair, skin or lips or (4) activity against parasites and insects. The invention also provides a method of making such a composition. The invention further provides a method of treating cancer, comprising administering to the host a composition comprising a therapeutically effective amount of a liposome comprising two or more agents (e.g., drugs or other active agents), wherein the combination of the two or more agents (e.g., drugs or other active agents) is cytotoxic to tumor cells, and a physiologically acceptable carrier. These and other advantages of the invention, as well as additional inventive features, will be apparent from the description of the invention provided herein.

### DETAILED DESCRIPTION OF THE INVENTION

The invention is directed to a composition comprising a physiologically acceptable carrier and two or more agents (e.g., drugs or other active agents) encapsulated into a liposome. Liposomes are well known in the art as spherical drug-delivery vehicles composed of a lipid bilayer (typically a phospholipid bilayer) surrounding an internal aqueous cavity (see, e.g., U.S. Pat. No. 6,146,659 and Published U.S. Patent Application No. 2003/0035830A1). The liposome according to the invention can be prepared using any suitable method known in the art. The chosen method will depend on the nature of the drugs or active agents (e.g., water-soluble, water-insoluble, or hydrophobic) encapsulated by the liposome. Standard methods for preparing liposomes are known to those skilled in the art, such as those described in, for example, U.S. Pat. Nos. 5,424,073, 5,648,090, and 6,146,659. In this regard, liposome preparation typically involves dissolving or dispersing lipophilic liposome-forming ingredients, such as those described herein, in a suitable solvent or combination of solvents and dried. Suitable solvents include any non-polar or slightly polar solvent, such as t-butanol, ethanol, methanol, chloroform, or acetone, that can be evaporated without leaving a pharmaceutically unacceptable residue. Drying can be by any suitable means such as by lyophilization. Hydrophilic ingredients can be dissolved in polar solvents, including water.

Liposomes typically are prepared by mixing the dried lipophilic ingredients with a polar, hydrophilic solution, preferably an aqueous solution. Suitable solutions include water or aqueous solutions containing pharmaceutically acceptable salts, buffers, or their mixtures. The liposomes are hydrated by dispersing the lipid in the aqueous solution with vigorous mixing. Any method of mixing can be used provided that the chosen method induces sufficient shearing forces between the lipid film and polar solvent to strongly homogenize the mixture and form the desired complexes. For example, mixing can be by vortexing, magnetic stirring, and/or sonicating. Where multilamellar liposomes are desired, they can be formed simply by vortexing the solution. Where unilamellar liposomes are desired, a sonication, filtration or extrusion step is included in the process.

Where active agents are included in the liposomes, they can be dissolved or dispersed in a suitable solvent and added to the liposome mixture prior to mixing. Typically, hydrophilic active agents are encapsulated into liposomes by hydrating the dry lipid film with an aqueous solution of the active agent (also referred to as simple encapsulation). In this manner, the active agent is passively encapsulated in the interlamellar spaces of the liposome. Alternatively, hydrophilic, water-soluble active agents can be encapsulated in liposomes by a reverse loading technique. This method involves the dispersal of neutrally charged drugs or other active agents in the aqueous phase of a liposome preparation, which allows the uncharged drugs or other active agents to permeate into liposomes via the lipid bilayer. The pH of the liposome solution is adjusted to create a charge on the active agent, rendering the active agent unable to pass back through the bilayer and into the external medium, thereby entrapping the active agent in the liposome. Lipophilic active agents (e.g., hydrophobic drugs or other active agents or water-insoluble drugs or other active agents) can be incorporated into liposomes by partitioning. In this regard, the active agent is dissolved along with the lipophilic ingredients in a suitable nonpolar solvent. The resulting solution can either be dried and mixed with a polar solvent as described above, or directly added to the aqueous phase and extracted. In this manner, the active agent is incorporated into the lipid portion of the liposome bilayer. In another alternative embodiment, the active agent could be dissolved in a third solvent or solvent mix and added to the mixture of polar solvent with the lipid film prior to homogenizing the mixture. While the foregoing methods for liposome preparation are preferred, any suitable method for preparing liposomes and encapsulating drugs or other active agents therein is within the scope of the present invention.

Desirably, the inventive composition comprises a liposome containing cardiolipin. Any suitable cardiolipin can be used in the present invention. For example, cardiolipin can be purified from natural sources or can be chemically synthesized, such as tetramyristylcardiolipin, by such methods as are known in the art. Liposome formulations containing cardiolipin are known in the art and are described in, for example, U.S. Pat. No. 6,146,659 and published U.S. Patent Application No. 2003/0035830 A1.

In embodiments where cardiolipin is present in the liposome of the inventive composition, the cardiolipin preferably comprises fatty acid chains of varying length and saturation. The basic structure of a phospholipid fatty acid comprises a hydrocarbon chain and a carboxylic acid group. In general, the length of the fatty acid hydrocarbon chain ranges from about 4 to about 30 carbon atoms; however, the carbon chain is more typically between about 12 and about 24 carbon atoms. In some embodiments, it is desirable for the hydrocarbon chain to comprise, for example, at least about 5 carbon atoms or at least about 10 carbon atoms or even at least about 15 carbon atoms. Typically, the length of the fatty acid hydrocarbon is less than about 30 carbon acids, such as less than about 25 carbon atoms, or even less than about 20 carbon atoms.

Most preferably, the cardiolipin used in the inventive composition comprises a short fatty acid chain (i.e., a “short-chain” cardiolipin). A short fatty acid chain comprises between about 4 and about 14 carbon atoms, and can have between about 6 and about 12 carbon atoms, such as between about 8 and about 10 carbon atoms. Alternatively, the cardiolipin can comprise a long fatty acid chain (i.e., a “long-chain” cardiolipin). A long fatty acid chain comprises between about 22 and about 30 carbon atoms, such as between about 24 and about 28 carbon atoms. The inventive composition is not limited to the use of short- or long-chain cardiolipin species exclusively. Indeed, a cardiolipin containing fatty acid chains of intermediate length can also be incorporated into the liposome of the invention.

The cardiolipin can be dissolved in a suitable solvent, which includes those in which cardiolipin is soluble and which: can be evaporated without leaving a pharmaceutically unacceptable residue. Non-polar or slightly polar solvents can be used, such as ethanol, methanol, chloroform, or acetone. In accordance with the present invention, separate solutions of cardiolipin and one or more drugs or other active agents can be mixed, or, alternatively, cardiolipin and one or more drugs or other active agents can be dissolved together in the same solution, as desired.

The inventive composition preferably comprises liposomes containing cardiolipin in combination with other lipophilic agents. Suitable lipophilic agents include pharmaceutically acceptable synthetic, semi-synthetic (modified natural) or naturally occurring compounds having a hydrophilic region and a hydrophobic region. Such compounds include amphiphilic molecules which can have net positive, negative, or neutral charges or which are devoid of charge. Suitable lipophilic agents include compounds, such as fatty acids and phospholipids which can be synthetic or derived from natural sources, such as egg or soy. Suitable phospholipids include compounds such as phosphatidylcholine (PC), phosphatidylethanolamine (PE), phosphatidylserine (PS), phosphatidylglycerol (PG), phosphatidic acid (PA), phosphatidylinositol (PI), sphingomyelin (SPM), and the like, alone or in combination. The phospholipids dimyristoylphosphatidylcholine (DMPC), dimyristoylphosphatidylglycerol (DMPG), dioleoylphosphatidylglycerol (DOPG), distearoylphosphatidyl choline (DSPC), dioleoylphosphatidylcholine (DOPC), dipalmitoylphosphatidylcholine (DPPC), diarachidonoyl phosphatidylcholine (DAPC), or hydrogenated soy phosphatidylcholine (HSPC) also can be used.

In accordance with the present invention, the liposomes can also include steroid components such as polyethylene glycol derivatives of cholesterol (PEG-cholesterols), coprostanol, cholestanol, or cholestane, or α-tocopherol. They may also contain sterol and sterol derivatives such as cholesterol hemisuccinate (CHS), cholesterol sulfate, and the like. Tocopherols and organic acid derivatives of tocopherols, such as α-tocopherol hemisuccinate (THS), can also be used. Suitable liposomes can also be formed with glycolipids, or natural or derivatized fatty acids and the like. The preferred liposome components include a mixture of cardiolipin, a phosphatidyl choline, cholesterol, and α-tocopherol.

In one preferred liposome composition, suitable amounts of two or more anticancer drugs or other active agents (as described herein), cardiolipin, cholesterol, phosphatidyl choline and α-tocopherol are combined. Suitable amounts of the two or more anticancer drugs or other active agents are those amounts that can be stably incorporated into the liposome of the present invention. In this regard, the two or more agents (e.g., drugs or other active agents) can each be present in the liposome in amounts from 1 to 50 wt. %, and more preferably 2 to 25 wt. %. The composition contains any suitable amount of cardiolipin including for example, from about 1 to 50 wt. %, about 2 to 25 wt. %, or about 5 to 20 wt. % cardiolipin. The inventive composition can contain any suitable amount of phosphatidylcholine including from about 1 to 95 wt. %, or about 20 to 75 wt. % phosphatidylcholine. Suitable amounts of α-tocopherol are from about 0.001 wt. % to about 5 wt. % α-tocopherol. For reference, wt. % refers to the relative mass of each ingredient in the final composition without regard to the amount of added water.

Generally, liposomes can have a net neutral, negative, or positive charge. For example, positive liposomes can be formed from a solution containing phosphatidylcholine, cholesterol, cardiolipin and enough stearylamine to overcome the net negative charge of cardiolipin or cationic variants of cardiolipin can be used. Negative liposomes can be formed from solutions containing phosphatidyl choline, cholesterol, and/or cardiolipin, for example.

The liposomes of the present invention can be multi or unilamellar vesicles depending on the particular composition and procedure used to make them. Liposomes can be prepared to have substantially homogeneous sizes in a selected size range. One effective sizing method involves extruding an aqueous suspension of the liposomes through a series of polycarbonate membranes having a selected uniform pore size. The pore size of the membrane will correspond roughly with the largest sizes of liposomes produced by extrusion through that membrane. For example, the liposomes can be formed and thereafter filtered through a 5 micron filter to obtain liposomes having a diameter of about 5 microns or less. Alternatively, 1 μm, 500 nm, 100 nm or other filters can be used to obtain liposomes having diameters of about 1 μm, 500 nm, 100 nm or any suitable size range, respectively. Alternatively, filtration can occur after formulation in liquid excipients or diluents, as hereinafter described.

Liposomes can be coated with a biodegradable polymer such as sucrose, epichlorohydrin, branched hydrophilic polymers of sucrose, polyethylene glycols, polyvinyl alcohols, methoxypolyethylene glycol, ethoxypolyethylene glycol, polyethylene oxide, polyoxyethylene, polyoxypropylene, cellulose acetate, sodium alginate, N,N-diethylaminoacetate, block copolymers of polyoxyethylene and polyoxypropylene, polyvinyl pyrrolidone, polyoxyethylene X-lauryl ether wherein X is from 9 to 20, and polyoxyethylene sorbitan esters.

Antioxidants can be included in liposomes. Suitable antioxidants include compounds such as ascorbic acid, tocopherol, and deteroxime mesylate.

Absorption enhancers can be included in liposomes. Suitable absorption enhancers include Na-salicylate-chenodeoxy cholate, Na deoxycholate, polyoxyethylene 9-lauryl ether, chenodeoxy cholate-deoxycholate and polyoxyethylene 9-lauryl ether, monoolein, Na tauro-24,25-dihydrofusidate, Na taurodeoxycholate, Na glycochenodeoxycholate, oleic acid, linoleic acid, linolenic acid. Polymeric absorption enhancers can also be included such as polyoxyethylene ethers, polyoxyethylene, sorbitan esters, polyoxyethylene 10-lauryl ether, polyoxyethylene 16-lauryl ether, azone (1-dodecylazacycloheptane-2-one).

The inventive composition can be used to administer virtually any drug or active agent to any suitable host (e.g., a human host). Suitable drugs include, for example, hydrophilic drugs, hydrophobic drugs, and water-insoluble drugs. A hydrophilic drug or other active agent is readily dissolved in water, and also is referred to in the art as “water-soluble.” A hydrophobic drug or other active agent has a low affinity for water, and does not readily dissolve in aqueous solutions. The dissolution of hydrophobic drugs or other active agents in water, however, is not impossible, and can be achieved under certain conditions that are known to those skilled in the art. Hydrophobic drugs or other active agents typically are dissolved in non-polar (e.g., lipophilic) solvents. In contrast, a water-insoluble drugs or other active agent cannot dissolve in water under any circumstances. In this regard, organic solvents typically are used to dissolve water-insoluble drugs or other active agents. In connection with the inventive composition, hydrophilic active agents can be included in the interior of the liposomes such that the liposome bilayer creates a diffusion barrier preventing it from randomly diffusing throughout the body. Hydrophobic or water-insoluble active agents are thought to be particularly well suited for use in the present composition because they not only benefit by exhibiting reduced toxicity but they tend to be well solubilized in the lipid bilayer of liposomes.

In accordance with the invention, the liposome preferably comprises two or more agents (e.g., drugs or other active agents). The two or more agents (e.g., drugs or other active agents) can be any combination of one or more hydrophobic agent(s), one or more water-insoluble agent(s), and/or one or more hydrophilic (i.e., water-soluble) agent. In this manner, each of the one or more hydrophilic agents is present in the aqueous cavity of the liposome, whereas each of the one or more hydrophilic (i.e., water-soluble) agents and/or water-insoluble agents is present in the lipid bilayer of the liposome. In a preferred embodiment of the invention, the liposome can comprise at least one hydrophilic (i.e., water-soluble) agent (e.g., drug or other active agent) and at least one water-insoluble agent (e.g., drug or other active agent). Alternatively, the liposome can comprise at least one hydrophilic (i.e., water-soluble) agent (e.g., drug or other active agent) and one hydrophobic agent (e.g., drug or other active agent). Most preferably, the liposome comprises one hydrophilic (i.e., water-soluble) agent (e.g., drug or other active agent) in combination with one water-insoluble agent (e.g., drug or other active agent) or one hydrophobic agent (e.g., drug or other active agent). Thus, in such liposome compositions, the water-soluble agent (e.g., drug or other active agent) is present in the aqueous cavity of the liposome, while the water-insoluble agent (e.g., drug or other active agent) or the hydrophobic agent (e.g., drug or other active agent) is present in the lipid bilayer of the liposome. In yet another alternative embodiment, the liposome preferably can comprise two or more agents (e.g., drugs or other active agents), each of which is hydrophilic (i.e., water-soluble). In this regard, each of the two or more agents (e.g., drugs or other active agents) is present in the aqueous cavity of the liposome, while no agent (e.g., drug or other active agent) are present in the lipid bilayer of the liposome. Still another preferred liposome composition comprises two or more water-insoluble or hydrophobic agents (e.g., drugs or other active agents). In such liposome compositions, each of the two or more agents (e.g., drugs or other active agents) is present in the lipid bilayer of the liposome, while no agents (e.g., drugs or other active agents) are present in the aqueous cavity of the liposome.

Desirably, the combination of agents (e.g., the two or more drugs or other active agents) is cytotoxic to a particular cell or cell type, and most preferably the combination is cytotoxic to tumor cells. In this respect, the combination of the two or more agents can include two or more drugs or other agents cytotoxic to tumor cells. In other embodiments, the combination of two or more agents exhibits activity against parasites and insects, such as skin-penetrating parasites and insects. For example, the agents can be insect or parasite repellants or insecticides or agents toxic to insects and parasites, such as are employed in the art. In other embodiments, the combination of agents can be suitable for application to nails, hair, skin, or lips. For example one or more of the two or more agents in the composition can be a cosmetic agent (such as a pigment or dye-containing colorant) suitable for coloring nails, hair, skin, lips, etc.). In other embodiments, the combination of the two or more agents can include drugs, nutritional supplements, vitamins, minerals, enzymes, hormones, proteins, and peptides, and, in such embodiments, one or more of the agents in the composition can be selected from such group. In another preferred embodiment, the combination of the two or more agents comprises at least one or more appetite suppressants, which can include any suitable agent for suppressing appetite, many of which are known in the art.

The drugs or other active agents incorporated into the inventive composition preferably are anticancer agents (e.g., chemotherapeutic agents), in that they are capable of inducing (either directly or indirectly) cancer cell or tumor cell cytotoxicity. Exemplary anticancer agents include mitoxantrone (see, e.g., international patent application publication WO 02/32400), taxanes (see, e.g., international patent application publications WO 01/70220 and WO 00/01366), paclitaxel, camptothecin, camptothecin derivaties (e.g., SN-38 (see, e.g., international patent application publications WO 02/058622 and WO 04/017940)), topotecan, gemcitabine (see, e.g., international patent application publication WO 04/017944), vinorelbine (see, e.g., international patent application publication WO 03/018018), vinblastine, anthracyclines, adria, adriamycin, adriamycine, capecitabine, doctaxel, doxorubicin, didanosine (ddl), stavudine (d4T), antisense oligonucleotides (e.g., c-raf antisense oligonucleotide (RafAON) (see, e.g., U.S. Pat. Nos. 6,126,965 and 6,559,129)), antibodies (e.g., herceptin), immunotoxins, hydroxyurea, melphalan, chlormethine, extramustinephosphate, uramustine, ifosfamide, mannomustine, trifosfamide, streptozotocin, mitobronitol, mitoxantrone, methotrexate, 5-fluorouracil, cytarabine, tegafur, idoxide, taxol, daunomycin, daunorubicin, bleomycin, amphotericin (e.g., amphotericin B), carboplatin, cisplatin, BCNU, vincristine, camptothecin, mitomycin, doxorubicin, etopside, histermine dihydrochloride, tamoxifen, cytoxan, leucovorin, oxaliplatin, irinotecan (see, e.g., international patent application publication WO 03/030864), 5-irinotecan, raltitrexed, epirubicin, anastrozole, proleukin, sulindac, EKI-569, erthroxylaceae, cerubidine, docetaxel, cytokines (e.g., interleukins, such as interleukin-2), ribozymes, interferons, oligonucleotides, and functional derivatives of the foregoing.

In a preferred embodiment of the invention, at least one of the two or more agents present in the inventive composition is a nucleic acid, such as a polynucleotide. Suitable polynucleotides include, for example, ribozymes, an interfering RNA (RNAi) or a antisense RNA or DNA sequence. In a preferred embodiment, the liposomal composition comprises an antisense oligonucleotide, typically comprising at least between about 7 and 13 nucleotides and up to between about 32 and 38 nucleotides (e.g., between about 10 and about 35 nucleotides) directed against a gene encoding a product that promotes tumor initiation and/or progression. A preferred antisense oligonucleotide targets c-raf (e.g., a c-raf antisense oligonucleotide (RafAON) such asone which includes, as at least part of its sequence, 5′-GTGCTCCATTGATGC-3′ (SEQ ID NO:1)). Where such oligonucleotides are included, the formulation also can include at least one drug, such as paclitaxel, mitoxantrone, camptothecins (preferably 7-ethyl-10-hydroxycamptothecin, i.e., SN-38) doxorubicin, gemcitabine, vinorelbine, vinblastine, cisplatin, 5-fluorouracil, mitomycin, and adriamycin. Alternatively, the inventive composition comprises a liposome comprising gemcitabine and at least one drug selected from the group consisting of cisplatin, carboplatin, paclitaxel, topotecan, doxorubicin, and vinorelbine. Other suitable drug combinations for use in the inventive composition include: (i) paclitaxel and carboplatin, (ii) irinotecan, paclitaxel, and carboplatin, (iii) irinotecan and raltitrexed, (iv) gemcitabine and epirubicin, (v) daunorubicin and doxorubicin, (vi) capecitabine and doctaxel, (vii) ddl, d4T, and hydroxyurea, (viii) vinorelbine and taxol, (ix) interleukin-2, histermine dihydrochloride, tamoxifen, and cisplatin, (x) herceptin and taxol, (xi) adriamycin, cytoxan, and herceptin, (xii) 5-fluorouracil, leucovorin, oxaliplaint, and irinotecan, (xiii) anastrozole and tamoxifen, (xiv) proleukin and herceptin, (xv) sulindac and EKI-569, and (xvi) erythroxylaceae and vinblastine. The inventive composition, however, is not limited to these exemplary anticancer drugs or to these specific combinations. Any combination of suitable anticancer agents can be used in connection with the inventive composition. Methods of using certain of the aforementioned drug combinations in non-liposomal formulations to treat cancer are known in the art and are described in, for example, Pathak et al., J. Am. Coll. Nutr., 21, 416-421 (2002), Socinski et al., Cancer, 95, 1520-1527 (2002), Lewis et al., Cancer Chemother. Pharmacol., 50, 257-265 (2002), Ricci et al., Cancer, 95, 1444-1450 (2002), Park et al., Breast Cancer Res., 4, 95-99 (2002), Thigpen, T., Semin. Oncol., 29(1 Suppl. 1), 11-16(2002), and U.S. Pat. No. 5,744,460.

Other drugs or active agents which are compatible with the present invention include agents which act on the peripheral nerves, adrenergic receptors, cholinergic receptors, the skeletal muscles, the cardiovascular system, smooth muscles, the blood circulatory system, synaptic sites, neuroeffector junctional sites, endocrine and hormone systems, the immunological system, the reproductive system, the skeletal system, the alimentary and excretory systems, the histamine system and the central nervous system. Suitable agents may be selected from, for example, proteins, enzymes, hormones, nucleotides, polynucleotides, nucleoproteins, polysaccharides, glycoproteins, lipoproteins, polypeptides, steroids, terpenoids, retinoids, anti-ulcer H2 receptor antagonists, antiulcer drugs, hypocalcemic agents, moisturizers, cosmetics, etc. Active agents can be analgesics; anesthetics; anti-arrythmic agents, antibiotics; antiallergic agents, antifingal agents, antihypertensive agents (e.g., dihydropyridines, antidepressants, cox-2 inhibitors); anticoagulants; antidepressants; antidiabetic agents, anti-epilepsy agents, antiinflammatory corticosteroids; agents for treating Alzheimers or Parkinson's disease; antiulcer agents; anti-protozoal agents, anxiolytics, thyroids, anti-thyroids, antivirals, anoretics, bisphosphonates, cardiac inotropic agents, cardiovascular agents, corticosteroids, diuretics, dopaminergic agents, gastrointestinal agents, hemostatics, hypercholesterol agents, antihypertensive agents; immunosuppressive agents; anti-gout agents, anti-malarials, anti-migraine agents, antimuscarinic agents, antiinflammatory agents, such as agents for treating rheumatology, arthritis, psoriasis, inflammatory bowel disease, Crohn's disease; or agents for treating demyelinating diseases including multiple sclerosis; ophthalmic agents; vaccines (e.g., against influenza virus, pneumonia, hepatitis A, hepatitis B, hepatitis C, cholera toxin B-subunit, typhoid, plasmodium falciparum, diphtheria, tetanus, herpes simplex virus, tuberculosis, HIV, bordetela pertusis, measles, mumps, rubella, bacterial toxoids, vaccinia virus, adenovirus, canary virus, bacillus calmette Guerin, klebsiella pneumonia vaccine, etc.); histamine receptor antagonists, hypnotics, kidney protective agents, lipid regulating agents, muscle relaxants, neuroleptics, neurotropic agents, opioid agonists and antagonists, parasympathomimetics, protease inhibitors, prostaglandins, sedatives, sex hormones (e.g., androgens, estrogens, etc.), stimulants, sympathominetics, vasodilators, xanthins, and synthetic analogs of these species.

The agents or drugs can be nephrotoxic, such as cyclosporins and amphotericin B, or cardiotoxic, such as amphotericin B and paclitaxel. Additional examples of drugs which may be delivered by way of the inventive composition include, prochlorperzine edisylate, ferrous sulfate, aminocaproic acid, mecamylamine hydrochloride, procainamide hydrochloride, amphetamine sulfate, methamphetamine hydrochloride, benzamphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, thiethylperzine maleate, anisindone, diphenadione erythrityl tetranitrate, digoxin, isoflurophate, acetazolamide, methazolamide, bendroflumethiazide, chloropromaide, tolazamide, chlormadinone acetate, phenaglycodol, allopurinol, aluminum aspirin, methotrexate, acetyl sulfisoxazole, erythromycin, hydrocortisone, hydrocorticosterone acetate, cortisone acetate, dexamethasone and its derivatives such as betamethasone, triamcinolone, methyltestosterone, 17-S-estradiol, ethinyl estradiol, ethinyl estradiol 3-methyl ether, prednisolone, 17α-hydroxyprogesterone acetate, 19-norprogesterone, norgestrel, norethindrone, norethisterone, norethiederone, progesterone, norgesterone, norethynodrel, aspirin, indomethacin, naproxen, fenoprofen, indoprofen, nitroglycerin, isosorbide dinitrate, propranolol, timolol, atenolol, alprenolol, cimetidine, clonidine, imipramine, levodopa, chlorpromazine, methyldopa, dihydroxyphenylalanine, theophylline, calcium gluconate, ketoprofen, ibuprofen, cephalexin, haloperidol, zomepirac, ferrous lactate, vincamine, diazepam, phenoxybenzamine, diltiazem, milrinone, mandol, quanbenz, hydrochlorothiazide, ranitidine, flurbiprofen, fenufen, fluprofen, tolmetin, alclofenac, mefenamic, flufenamic, difuinal, nimodipine, nitrendipine, nisoldipine, nicardipine, felodipine, lidoflazine, tiapamil, gallopamil, amlodipine, mioflazine, lisinolpril, enalapril, enalaprilat captopril, ramipril, famotidine, nizatidine, sucralfate, etintidine, tetratolol, minoxidil, chlordiazepoxide, diazepam, amitriptyline, and imipramine. Further examples are proteins and peptides which include, but are not limited to, bone morphogenic proteins, insulin, heparin, colchicine, glucagon, thyroid stimulating hormone, parathyroid and pituitary hormones, calcitonin, renin, prolactin, corticotrophin, thyrotropic hormone, follicle stimulating hormone, chorionic gonadotropin, gonadotropin releasing hormone, somatotropins (e.g., bovine somatotropin, porcine somatotropin, etc.), oxytocin, vasopressin, GRF, somatostatin, lypressin, pancreozymin, luteinizing hormone, LHRH, LHRH agonists and antagonists, leuprolide, interferons (e.g., α-, β-, or γ-interferon, interferon α-2a, interferon α-2b, and consensus interferon, etc.), interleukins, growth hormones (e.g., human growth hormone and its derivatives such as methione-human growth hormone and des-phenylalanine human growth hormone, bovine growth hormone, porcine growth hormone, insulin-like growth hormone, etc.), fertility inhibitors such as the prostaglandins, fertility promoters, growth factors such as insulin-like growth factor, coagulation factors, pancreas hormone releasing factor, analogs and derivatives of these compounds, and pharmaceutically acceptable salts of these compounds, or their analogs or derivatives.

The invention further provides a method of preparing liposomes containing a plurality of active agents, such as herein described. In accordance with this aspect of the invention, the method comprises first formulating a liposomal preparation comprising at least one initial active agent. To this initial preparation, at least one additive active agent (e.g., second active agent) is added. The additive active agent can be added, for example, by including it (e.g., by dissolving it or suspending it) in a hydrating solution (typically an aqueous solution), which can be used to reconstitute a lyophilized preparation (i.e., a “cake”) containing a liposomal preparation comprising at least one initial active agent. The initial active agent and the additive active agent can be any desired active agents, such as those described elsewhere herein.

Where the additive active agent is added shortly before administration, the method facilitates greater therapeutic success upon administration of the agent to a human or animal patient. For example, where the initial formulation includes a liposomal formulation of paclitaxel (LEP) (the initial active agent comprises paclitaxel), an additive active agent, such as mitoxantrone, anthracycline, or doxorubicin, can be employed as the additive agent, which is added to the initial formulation prior to administration. Where the initial formulation includes a liposomal formulation of SN-38 (i.e., the initial agent comprises SN-38), an additive active agent, such as gemcitabine, can be added to the initial formulation prior to administration to a human or animal patient. In this context, the additive active agent is added prior to administration to the patient in the sense that the formulation is readied for administration at or near the time of inclusion of the additive active agent, i.e., the formulation is not thereafter further processed for storage. Typically, the formulation is administered to a patient within one or a few hours following addition of the additive active agent, and more typically the formulation is administered to a patient fairly soon (e.g., less than about 30 minutes, and more typically only a few minutes) following inclusion of the additive active agent. In this sense, the inventive method in accordance with this aspect of the invention facilitates bedside preparation of a liposomal formulation that can lead to enhanced efficacy when treating a human or animal patient.

Other preferred agents, which can serve as either the initial or the additive agent in accordance with the inventive method include nucleic acids, such as polynucleotides. Preferred polynucleotides for use as the initial or additive agent include ribozymes, interfering RNAs (RNAi) or an antisense RNA or DNA oligonucleotides, such as antisense oligonucleotides. A particular preferred antisense oligonucleotide is antisense to c-raf, such as herein described and otherwise known in the art.

The initial or the additive agent also can be one or more agents such as agents for treating Alzheimers or Parkinson's disease, agents for treating Crohn's disease, agents for treating demyelinating diseases including multiple sclerosis, agents for treating rheumatology, analgesics, anastrozole, anesthetics, anoretics, anthracyclines, antiallergic agents, anti-arrythmic agents, antibiotics, antibodies, anticoagulants, antidepressants, antidiabetic agents, anti-epilepsy agents, antifungal agents, anti-gout agents, antihypertensive agents, antiinflammatory agents, antiinflammatory corticosteroids, anti-malarials, anti-migraine agents, antimuscarinic agents, anti-protozoal agents, antisense oligonucleotides, anti-thyroids, antiulcer agents, antiulcer drugs, anti-ulcer H2 receptor antagonists, antivirals, anxiolytics, agents for treating arthritis, bisphosphonates, bone morphogenic proteins, camptothecins, cardiac inotropic agents, cardiovascular agents, coagulation factors, corticosteroids, cosmetics, cox-2 inhibitors, cyclosporins, cytokines, derivatives of dexamethasone, dihydropyridines, diuretics, dopaminergic agents, fertility inhibitors, fertility promoters, gastrointestinal agents, glycoproteins, growth factors and hormones, derivatives of human grown hormone, hemostatics, histamine receptor antagonists, hypercholesterol agents, hypnotics, hypocalcemic agents, immunosuppressive agents, immunotoxins, agents for treating inflammatory bowel disease, interferons, interleukins, kidney protective agents, LHRH agonists and antagonists, lipid regulating agents, lipoproteins, moisturizers, muscle relaxants, nephrotoxins, neuroleptics, neurotropic agents, nucleoproteins, nucleotides, oligonucleotides, enzymes, hormones, ophthalmic agents, opioid agonists and antagonists, parasympathomimetics, parathyrQid and pituitary hormones, polynucleotides, polypeptides, polysaccharides, prostaglandins, protease inhibitors, proteins, agents for treating psoriasis, retinoids, ribozymes, sedatives, sex hormones, somatostatin, somatotropins, steroids, stimulants, sympathomimetics, taxanes, terpenoids, thyroids, vaccines, and vasodilators. Of course, any of these agents also can be used as one or more of the two or more active agents included in the inventive composition, as desired.

Alternatively, or in addition, the initial or the additive agent included in accordance with the inventive method can be one or more agents such as 17α-hydroxyprogesterone acetate, 17-S-estradiol, 19-norprogesterone, 5-fluorouracil, 5-irinotecan, acetazolamide, acetyl sulfisoxazole, adria, adriamycin, adriamycine, alclofenac, allopurinol, alprenolol, aluminum aspirin, aminocaproic acid, amitriptyline, amlodipine, amphetamine sulfate, amphotericin, amphotericin B, anisindone, herceptin, aspirin, atenolol, atropine sulfate, BCNU, bendroflumethiazide, benzamphetamine hydrochloride, bethanechol chloride, bleomycin, calcitonin, calcium gluconate, SN-38, capecitabine, carboplatin, cephalexin, cephalexin hydrochloride, cerubidine, chlordiazepoxide, chlormadinone acetate, chlormethine, chloropromaide, chlorpromazine, chorionic gonadotropin, cimetidine, cisplatin, clonidine, colchicine, corticotrophin, cortisone acetate, cytarabine, cytoxan, cytoxin, daunomycin, daunorubicin, dexamethasone, betamethasone, diazepam, didanosine (ddl), difuinal, digoxin, dihydroxyphenylalanine, diltiazem, diphenadione erythrityl tetranitrate, diphenidol, docetaxel, doctaxel, doxorubicin (including pegylated doxorubicin), EKI-569, enalapril, enalaprilat captopril, epirubicin, erthroxylaceae, erythromycin, erythroxylaceae, Erythroxylacease, ethinyl estradiol, ethinyl estradiol 3-methyl ether, etintidine, etopside, extramustinephosphate, famotidine, felodipine, fenoprofen, fenufen, ferrous sulfate, flufenamic, fluprofen, flurbiprofen, follicle stimulating hormone, gallopamil, gemcitabine, glucagon, gonadotropin releasing hormone, human growth hormone, methione-human growth hormone, des-phenylalanine human growth hormone, bovine growth hormone, porcine growth hormone, insulin-like growth hormone, haloperidol, heparin, herceptin, histermine dihydrochloride, hydrochlorothiazide, hydrocorticosterone acetate, hydrocortisone, hydroxyurea, ibuprofen, idoxide, ifosfamide, imipramine, indomethacin, indoprofen, insulin, insulin-like growth factor, α-interferon, β-interferon, γ-interferon, interferon α-2a, interferon α-2b, consensus interferon, interleukin-2, irinotecan, irinotecan sulindac, isoflurophate, isopropamide iodide, isoproterenol sulfate, isosorbide dinitrate, ketoprofen, leucovorin, leuprolide, levodopa, LHRH, lidoflazine, lisinolpril, luteinizing hormone, lypressin, mandol, mannomustine, mecamylamine hydrochloride, meclizine hydrochloride, mefenamic, melphalan, methacholine chloride, methamphetamine hydrochloride, methazolamide, methotrexate, methyldopa, methylphenidate hydrochloride, methyltestosterone, milrinone, minoxidil, mioflazine, mitobronitol, mitomycin, mitoxantrone, naproxen, nicardipine, nimodipine, nisoldipine, nitrendipine, nitroglycerin, nizatidine, norethiederone, norethindrone, norethisterone, norethynodrel, norgesterone, norgestrel, oxaliplatin, oxytocin, paclitaxel, pancreas hormone releasing factor, pancreozymin, phenaglycodol, phenformin hydrochloride, phenmetrazine hydrochloride, phenoxybenzamine, pilocarpine hydrochloride, prednisolone, procainamide hydrochloride, prochlorperazine maleate, prochlorperzine edisylate, progesterone, prolactin, proleukin, propranolol, quanbenz, raltitrexed, ramipril, ranitidine, reltitrexed, renin, androgens, estrogens, scopolamine bromide, bovine somatotropin, porcine somatotropin, stavudine (d4T), streptozotocin, sucralfate, sulindac, tamoxifen, taxol, tegafur, tetratolol, theophylline, theophylline cholinate, thiethylperzine maleate, thyroid stimulating hormone, thyrotropic hormone, tiapamil, timolol, tolazarnide, tolmetin, topotecan, triamcinolone, tridihexethyl chloride, trifosfamide, uramustine, vasopressin, vinblastine, vincamine, vincristine, vinorelbine, xanthins, and zomepirac, and a vaccine against influenza virus, pneumonia, hepatitis A, hepatitis B, hepatitis C, cholera toxin B-subunit, typhoid, plasmodium falciparum, diphtheria, tetanus, herpes simplex virus, tuberculosis, HIV, bordetela pertusis, measles, mumps, rubella, bacterial toxoids, vaccinia virus, adenovirus, canary virus, bacillus calmette, Guerin, or klebsiella pneumonia. Of course, any of these agents also can be used as one or more of the two or more active agents included in the inventive composition, as desired.

The liposome of the inventive composition can be conjugated to a targeting agent that directs binding of the liposome to a tumor cell. Targeting agents can be bound to the liposome such that the liposome can be targeted to particular tissues or organs. The agents can be bound through covalent, electrostatic, or hydrophobic bonds with the complexes. Suitable targeting agents include proteins and carbohydrates or other agents as are known to target desired tissues or organs. Suitable protein targeting agents include, for example, antibodies, antibody fragments, peptides, peptide hormones, receptor ligands, and mixtures thereof. Suitable carbohydrate targeting moieties include polysaccharides. U.S. Pat. No. 6,056,973 discloses a number of targeting agents and target cells (see, e.g., col. 11, lines 1-41), and methods of preparing suitable conjugates (see, e.g., col. 11, line 55 -col. 14, line 20).

The invention provides a method of treating cancer in a mammalian host, comprising administering to the host a composition comprising (i) a therapeutically effective amount of a liposome comprising two or more agents (e.g., drugs or other active agents), wherein the combination of the two or more agents (e.g., drugs or other active agents) is cytotoxic to tumor cells, and (ii) a physiologically acceptable carrier. Descriptions of the liposome, active agents contained therein, liposome targeting agents, and components thereof set forth above in connection with other embodiments of the invention are applicable to those same aspects of the aforesaid inventive method.

Ideally, the inventive method is used to treat a cancer manifested as a solid tumor or a tumor associated with soft tissue (i.e., soft tissue sarcoma) in a human. The tumor can be associated with cancers of (i.e., located in) the oral cavity and pharynx, the digestive system, the respiratory system, bones and joints (e.g., bony metastases), soft tissue, the skin (e.g., melanoma), breast, the genital system, the urinary system, the eye and orbit, the brain and nervous system (e.g., glioma), or the endocrine system (e.g., thyroid) and is not necessarily the primary tumor. Tissues associated with the oral cavity include, but are not limited to, the tongue and tissues of the mouth. Cancer can arise in tissues of the digestive system including, for example, the esophagus, stomach, small intestine, colon, rectum, anus, liver, gall bladder, and pancreas. Cancers of the respiratory system can affect the larynx, lung, and bronchus and include, for example, non-small cell lung carcinoma. Tumors can arise in the uterine cervix, uterine corpus, ovary vulva, vagina, prostate, testis, and penis, which make up the male and female genital systems, and the urinary bladder, kidney, renal pelvis, and ureter, which comprise the urinary system. The target tissue also can be associated with lymphoma (e.g., Hodgkin's disease and Non-Hodgkin's lymphoma), multiple myeloma, or leukemia (e.g., acute lymphocytic leukemia, chronic lymphocytic leukemia, acute myeloid leukemia, chronic myeloid leukemia, and the like).

The tumor can be at any stage, and can be subject to other therapies. The inventive method is useful in treating tumors that have been proven to be resistant to other forms of cancer therapy, such as radiation-resistant tumors. The tumor also can be of any size.

In the context of the inventive method, a therapeutically effective amount of the liposome composition is administered to a mammalian host, most preferably a human host. A “therapeutically effective amount” means an amount sufficient to show a meaningful benefit in an individual, i.e., promoting at least one aspect of tumor cell cytotoxicity, or treatment, healing, prevention, or amelioration of other relevant medical condition(s) associated with a particular cancer. Therapeutically effective amounts may vary depending upon the biological effect desired in the individual, cancer to be treated, and/or the specific characteristics of the liposome composition (or drugs encapsulated therein), and individual. Thus, the attending physician (or other medical professional responsible for administering the composition) will typically decide the amount of liposome composition with which to treat each individual patient

The liposome composition preferably is included in a pharmaceutical preparation in dosage units. This means that the preparations are in the form of individual parts, for example capsules, pills, suppositories and ampoules, of which the content of the liposome composition corresponds to a fraction or a multiple of an individual dose. The dosage units can contain, for example, 1, 2, 3 or 4 individual doses or a fraction of (e.g., ½, ⅓, or ¼, etc.) of an individual dose. An individual dose preferably contains the amount of the liposome which is given in one administration and which usually corresponds to a whole, a half, a third, or a quarter of a daily dose. In this regard, the liposome should preferably be present in a pharmaceutical preparation at a concentration of about 0.01 to 5 wt. %, about 0.05 to 1 wt. %, about 0.1 to 1.5 wt. %, about 0.2 to 1 wt. %, or about 0.5 to 1 wt. % relative to the total mixture. However, it can be necessary to deviate from the dosages mentioned and in particular to do so as a function of the nature and body weight of the subject to be treated, the nature and the severity of the illness, the nature of the preparation and if the administration of the medicine, and the time or interval over which the administration takes place. Thus it can suffice in some cases to manage with less that the abovementioned amount of active compound, whilst in other cases the abovementioned amount of active compound must be exceeded. The particular required optimum dosage and the type of administration of the liposome composition can be determined by one skilled in the art, by available methods. Suitable amounts are therapeutically effective amounts that do not have excessive toxicity, as determined in empirical studies.

In addition to its cytotoxic effect on tumor cells, the inventive composition also provides a means by which multidrug resistance can be modulated in tumor cells subject to standard, non-liposomal forms of chemotherapy. In particular, the present compositions reduce the tendency of cancer cells subjected to combination chemotherapy to develop resistance thereto.

In accordance with the inventive method, the liposome composition desirably is formulated into a pharmaceutical composition comprising a physiologically acceptable (e.g., a pharmaceutically or pharmacologically acceptable) carrier (e.g., excipient or diluent). Any suitable physiologically acceptable carrier can be used within the context of the invention, and such carriers are well known in the art. Most preferably, the inventive method employs a non-toxic, inert physiologically-acceptable carrier. Such carriers are known in the art and include, for example, semi-solid or liquid diluents, fillers and formulation auxiliaries of all kinds. The carrier typically will be liquid, but also can be solid, or a combination of liquid and solid components. The choice of carrier will be determined, at least in part, by the location of the target tissue and/or cells, and the particular method used to administer the composition.

Typically, such compositions can be prepared as injectables, either as liquid solutions or suspensions. Solid forms suitable for using to prepare solutions or suspensions upon the addition of a liquid prior to injection can also be prepared, and the preparations can also be emulsified. The pharmaceutical forms suitable for injectable use include sterile aqueous solutions or dispersions, formulations including sesame oil, peanut oil or aqueous propylene glycol, and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. Solutions of the active compounds as free base or pharmacologically acceptable salts can be prepared in water suitably mixed with a surfactant, such as hydroxycellulose. Dispersions can also be prepared in glycerol, liquid polyethylene glycols, and mixtures thereof and in oils. Under ordinary conditions of storage and use, these preparations contain a preservative to prevent the growth of microorganisms.

The liposome for use in the present invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such as organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

The composition can further comprise any other suitable components, especially for enhancing the stability of the composition and/or its end-use. Accordingly, there is a wide variety of suitable formulations of the composition of the invention. The following formulations and methods are merely exemplary and are in no way limiting.

For oral administration, the liposome composition can be formulated as tablets, capsules, lozenges, powders, syrups, aqueous solutions, suspensions, and the like. Carriers such as lactose, sodium citrate, and salts of phosphoric acid can be used to prepare tablets. Further, disintegrants such as starch, and lubricating agents, such as magnesium stearate, sodium lauryl sulfate and talc can be included. Diluents such as lactose and high molecular weight polyethylene glycols can be used in the preparation of dosages in capsule form. The active ingredient can be combined with emulsifying and suspending agents to generate aqueous suspensions for oral use. Flavoring agents such as sweeteners can be added, as desired.

For topical (e.g., dermal) administration, the liposome composition can be provided in the form of gels, oils, and emulsions by the addition of suitable water-soluble or water-insoluble excipients, for example polyethylene glycols, certain fats, and esters or mixtures of these substances. Suitable excipients are those in which the liposome composition is sufficiently stable to allow for therapeutic use. Such formulations also have particular applicability where the combination of two or more agents in the composition is for application to nails, hair, skin or lips, or wherein the combination of the two or more agents is a cosmetic. In such embodiments, the composition can be formulated for application as lipsticks or pencils, nail polish, hair gels or sprays, powders, creams, and other formulations employed for cosmetic application.

Formulations suitable for anal administration can be prepared as suppositories by mixing the active ingredient with a variety of bases such as emulsifying bases or water-soluble bases. Formulations suitable for vaginal administration can be presented as pessaries, tampons, creams, gels, pastes, foams, or spray formulas containing, in addition to the active ingredient, such carriers as are known in the art to be appropriate.

Formulations suitable for administration via inhalation include aerosol formulations. The aerosol formulations can be placed into pressurized acceptable propellants, such as dichlorodifluoromethane, propane, nitrogen, and the like. They also can be formulated as non-pressurized preparations, for delivery from a nebulizer or an atomizer.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind previously described. In a preferred embodiment of the invention, the liposome composition is formulated for injection. In this regard, the formulation desirably is suitable for intratumoral administration, but also can be formulated for intravenous injection, intraperitoneal injection, subcutaneous injection, and the like. In this manner, for example, liposome formulations containing two or more anticancer drugs may be injected directly into tumor tissue for delivery of the anticancer drugs directly to cancer cells. In some cases, particularly after resection of a tumor, the liposome formulation can be implanted directly into the resulting cavity or may be applied to the remaining tissue as a coating. In cases in which the liposome formulation is administered after surgery, it is possible to utilize liposomes having larger diameters of about 1 micron since they do not have to pass through the vasculature.

In addition to the active agents, the liposome can comprise additional therapeutic or biologically-active agents. For example, therapeutic factors useful in the treatment of a particular indication can be present. Factors that control inflammation, such as ibuprofen or steroids, can be part of the composition to reduce swelling and inflammation associated with in vivo administration of the liposome composition and physiological distress. Immune system suppressors can be administered with the composition to reduce any immune response to the antibody itself or associated with a disorder. Alternatively, immune enhancers can be included in the composition to upregulate the body's natural defenses against disease. Moreover, cytokines can be administered with the composition to attract immune effector cells to a disease (e.g., tumor) site.

One preferred embodiment of the present invention includes a liposomal formulation comprising an oligonucleotide antisense to cRAF and paclitaxel as active agents. Another preferred embodiment includes a liposomal formulation comprising an oligonucleotide antisense to cRAF and mitoxantrone as active agents. Another preferred embodiment includes a liposomal formulation comprising an oligonucleotide antisense to cRAF and paclitaxel as active agents. Another preferred embodiment includes a liposomal formulation comprising an oligonucleotide antisense to cRAF and 7-ethyl-10-hydroxycamptothecin (SN-38) as active agents. Another preferred embodiment includes a liposomal formulation comprising an oligonucleotide antisense to cRAF and Gemcitabine as active agents. Another preferred embodiment includes a liposomal formulation comprising an oligonucleotide antisense to cRAF and vinorelbine as active agents. Another preferred embodiment includes a liposomal formulation comprising irinotecan and reltitrexed as active agents. Another preferred embodiment includes a liposomal formulation comprising daunorubicin and pegylated doxorubicin as active agents. Another preferred embodiment includes a liposomal formulation comprising an oligonucleotide and vinblastine, cisplatin, 5-fluorouricil, mitomycin, adriamycine as active agents or combinations thereof (e.g., an oligonucleotide, vinblastine, and adriamycine or oligonucleotide, 5-flurorouricil, and adriamycine as active agents). Another preferred embodiment includes a liposomal formulation comprising capecitabine and doctaxel as active agents. Another preferred embodiment includes a liposomal formulation comprising ddl, d4T (Stavudine) and hydroxyurea as active agents. Another preferred embodiment includes a liposomal formulation comprising vinorelbine and taxol as active agents.

A liposomal formulation comprising paclitaxel and carboplatin as active agents is suitable for treating lung cancers. A liposomal formulation comprising two or more agents selected from irinotecan, paclictaxel, and carboplatin as active agents also is useful for treating patients with lung cancers, particularly non-small cell lung carcinoma. Such formulations can be used to treat such cancers in accordance with the inventive method.

A liposomal formulation comprising gemcitabine and epirubicin as active agent is particularly useful for treating patients with urothelial carcinoma. Such formulations can be used to treat such cancers in accordance with the inventive method.

Formulations particularly useful for treating ovarian carcinoma include a liposomal formulation comprising gemcitabine and cisplatin as active agents; a liposomal formulation comprising gemcitabine and carboplatin as active agents; a liposomal formulation comprising gemcitabine and paclitaxel as active agents; a liposomal formulation comprising gemcitabine and topotecan as active agents and a liposomal formulation comprising gemcitabine and doxorubicin as active agents. Thus, embodiments of the present invention in which the two or more agents of the formulation are selected from the group consisting of gemcitabine, cisplatin, carboplatin, paclitaxel, topotecan, and doxorubicin can be used to treat ovarian carcinoma in accordance with the present invention.

A preferred embodiment particularly suitable for treatment of melanoma includes a liposomal formulation comprising interleukin-2 and histermine dihydrochloride as active agents. Another preferred embodiment suitable for treatment of melanoma includes a liposomal formulation comprising tamoxifen, and cisplatin as active agents. Thus, embodiments in which the two or more agents of the formulation are selected from the group consisting of interleukin-2, histermine dihydrochloride, tamoxifen and cisplatin can be used to treat melanoma in accordance with the present invention.

A preferred embodiment suitable for treatment of breast cancer includes a liposomal formulation comprising herceptin and paclitaxel as active agents. Another preferred embodiment suitable for treatment of breast cancer includes a liposomal formulation comprising adriamycin, cytoxin, and herceptin as active agents. Another preferred embodiment suitable for treatment of breast cancer includes a liposomal formulation comprising anastrozole and tamoxifen as active agents. Another preferred embodiment suitable for treatment of breast cancer includes a liposomal formulation comprising proleukin and herceptin as active agents. Thus, embodiments in which the two or more agents of the formulation are selected from the group consisting of herceptin, paclitaxel, adriamycin, cytoxin, anastrozole, tamoxifen and proleukin can be used to treat breast cancer in accordance with the present invention.

A preferred embodiment suitable for treatment of colorectal cancer includes a liposomal formulation comprising 5-flurouricil, leucovorin, and oxaliplatin as active agents. Another preferred embodiment suitable for treatment of colorectal cancer includes a liposomal formulation comprising 5-irinotecan, 5-flurouricil, and leucovorin as active agents. Another preferred embodiment suitable for treatment of colorectal cancer includes a liposomal formulation comprising oxaliplatin and irinotecan as active agents. Another preferred embodiment suitable for treatment of colon cancer includes a liposomal formulation comprising sulindac and EKI-569 as active agents. Thus, embodiments in which the two or more agents of the formulation are selected from the group consisting of 5-flurouricil, leucovorin, oxaliplatin, 5-irinotecan, irinotecan, sulindac and EKI-569 can be used to treat colorectal cancer in accordance with the present invention.

Another preferred embodiment includes a liposomal formulation comprising erythroxylacease and vinblastine as active agents. Erythroxylacease is a chloroform-soluble extract of the madacascan plant, Erythroxylum pervilli, which has been shown to modulate multidrug resistence.

The abovementioned pharmaceutical preparations are manufactured in the usual manner according to known methods, for example by mixing the liposome composition with the excipient or excipients.

### EXAMPLE 1

### Preparation of Liposomes Containing Cardiolipin Analogs

Liposomal doxorubicin was prepared for clinical administration by simple vortex mixing of a vial containing 40 mg of cardiolipin-liposome lyophilizate and 2.5 ml of a doxorubicin solution previously prepared in 0.85% NaCl at 2 mg/nl. Vortex mixing was performed for 1 minute, and the mixture was kept at 37° C. for a 15-minute incubation period.

### EXAMPLE 2

### Loading Multiple Active Agents in a Single Liposomal Formulation

To begin with, an initial formulation of liposomal encapsulated paclitaxel (LEP) was prepared; this preparation consisted of phosphatidylcholine, cholesterol and cardiolipin. Sucrose and tocopherol were added to the formulation as stablizers in order to form a sterilized lyophilized cake.

Either doxorubicin (0.5 to 1.5 mg/ml) or mitoxantrone (0.5 to 1.5 mg/ml) was dissolved in deionized water, and these solutions were employed to reconstitute the lyophilized LEP cakes. The drug to lipid ratio varied from 1:120 to 1:24 (wt/wt) for doxorubicin and 1:120 to 1:24 (wt/wt) for mitoxantrone. Following reconstitution, the liposomal preparation was subjected to column chromatography using Sephardex G-25 to separate the free doxorubicin or mitoxantrone from the drug bound to the liposomes. The pre and post column samples were solubilized in methanol and the absorbance values were measured at appropriate wavelengths. The absorbance of doxorubicin (480 nm) and mitoxantrone (660 nm) was measured in the formulations both prior to and after column chromatography, and the percent entrapment was calculated using the following equation:
% entrapment=[absorbance of drug after column/absorbance of drug before column]×100

The results of this assessment are presented in Table 1 for doxorubicin (DOX) and Table 2 for mitoxantrone (MTO).

TABLE 1

| Initial DOX Concentration | DOX % Entrapment |
|---|---|
| (mg/mL) | UV 480 nm |
| 0.50 | 93 |
| 0.75 | 92 |
| 1.00 | 90 |
| 1.50 | 78 |

TABLE 2

| Initial MTO Concentration | MTO % Entrapment |
|---|---|
| (mg/mL) | UV 660 nm |
| 0.50 | 97 |
| 0.75 | 99 |
| 1.00 | 100 |
| 1.50 | 101 |

Mitoxantrone or doxorubicin (0.5 to 1.5 mg) was loaded into LEP-ETU. The effect of mitoxantrone or doxorubicin loading on entrapment of paclitaxel and liposomal size are shown in Tables 3 and 4 respectively.

TABLE 3

| Initial Concentration MTO | Paclitaxel Entrapment (%) | |
|---|---|---|
| or DOX (mg/mL) | With MTO | With DOX |
| 0.50 | 101 | 102 |
| 0.75 | 96 | 102 |
| 1.00 | 97 | 102 |
| 1.50 | 105 | 109 |

TABLE 4

| Initial | | |
|---|---|---|
| Concentration of | | |
| MTO or DOX | Mean Size (nm) | |
| (mg/mL) | MTO in LEP | DOX in LEP |
| 0.50 | 125 | 133 |
| 0.75 | 128 | 130 |
| 1.00 | 127 | 131 |
| 1.50 | 129 | 129 |

From this study, it can be concluded that reconstitution of the LEP cake with doxorubicin or mitoxantrone solution resulted in entrapment of either of the additive drugs (doxorubicin or mitoxantrone) into the liposomal formulation of paclitaxel (LEP). Moreover, 78 to 100% of the additive drug was entrapped into the LEP at a drug to lipid ratio 1:120 to 1:15 for mitoxantrone and 1:120 to 1:24 for doxorubicine. Presence of an additional drug, doxorubicin or mitoxantrone, did not alter entrapment efficiency of paclitaxel in LEP-ETU, size of LEP-ETU or stability of LEP-ETU. Paclitaxel content remained intact after entrapping mitoxantrone or doxorubicin. This suggested that both drugs can co-exist in a single delivery system without compromising size, entrapment efficiency or stability of the liposomal formulation.

### EXAMPLE 3

### Stability Studies of LEP-DOX

The chemical stability of a LEP-DOX suspension was studied up to 120 hours post-reconstitution at refrigerator temperatures (2-8° C.) and at room temperature (25° C.). Stability parameters, such as particle size, lipid contents and paclitaxel and doxorubicin concentrations, were determined as a function of time. Mean vescicle diameter and sample size distribution were measured by a dynamic light scattering technique using Nicomp Model 380 Sub-micron Particle Sizer (Particle Sizing Systems, Santa Barbara Calif.). HPLC was used for quantification of the lipid and active components. As shown in Tables 5 and 6, there was no significant change for either particle size or initial concentrations of all of the components. These observations suggested the absence of aggregation in the liposome.

TABLE 5

| Time | Percent (%) of initial at 2-8° C. | | | | | Particle | Entrapment (%) | |
|---|---|---|---|---|---|---|---|---|
| in hrs | DOPC | Chol | Cardiolipin | Paclitaxel | DOX | Size (nm) | Paclitaxel | DOX |
| 0 | 100 | 100 | 100 | 100 | 100 | 110 | 100 | 100 |
| 24 | 102 | 101 | 101 | 96 | n/a | 109 | 97 | n/a |
| 48 | 99 | 97 | 100 | 94 | n/a | 111 | 99 | n/a |
| 72 | 99 | 98 | 106 | 93 | 96 | 111 | 99 | 97 |
| 120 | 100 | 100 | 117 | 93 | 100 | 111 | 95 | 97 |

TABLE 6

| Time | Percent (%) of initial at 25° C. | | | | | Particle | Entrapment (%) | |
|---|---|---|---|---|---|---|---|---|
| in hrs | DOPC | Chol | Cardiolipin | Paclitaxel | DOX | Size (nm) | Paclitaxel | DOX |
| 0 | 100 | 100 | 100 | 100 | 100.0 | 110 | 100 | 100 |
| 24 | 102 | 101 | 102 | 99 | n/a | 111 | 100 | n/a |
| 48 | 98 | 97 | 101 | 95 | n/a | 110 | 102 | n/a |
| 72 | 99 | 98 | 105 | 92 | 90.6 | 110 | 99 | 111 |
| 120 | 98 | 98 | 113 | 95 | 98.1 | 110 | 97 | 96 |

### EXAMPLE 4

### Cytoxicity of LEP-DOX Against Resistant Human Ovarian and Murine Leukemia Cell Lines

SKVLB (Vincristine-resistant human ovarian) cells were obtained from Georgetown University and maintained in RPMI 1640 medium containing 10% heat-inactivated FBS, penicillin (100 units/mL) and streptomycin (100 mg/mL) with 2 mM Vincristine. P388/ADR (Adriamycin-resistant murine leukemia) cells were purchased from National Cancer Institute (Frederick, Md.) and maintained in RPMI 1640 medium containing 10% heat-inactivated FBS, penicillin (100 units/mL) and streptomycin (100 mg/mL). SKVLB cells were cultured in drug-free media for at least a week before studies. The cells (10,000 cells/well for SKVLB and 25,000 cells/well for P388/ADR) were plated in a 96-well plate overnight and treated with doxorubicin, LEP or LEPD for 48 hrs. After incubation, the cytotoxicity was determined by a sulforhodamine B assay. Table 7 shows the enhanced cytotoxicity of LEP-DOX against the human ovarian and murine leukemia cell lines.

TABLE 7

| | GI50 values (mM) against drug-resistant cancer | |
|---|---|---|
| | cell lines | |
| | SKVLB | |
| | (Vincristine- | P388/ADR |
| | resistant human | (Adriamycin-resistant |
| Formulations | ovarian) | murine leukemia) |
| Doxorubicin (DOX) | 16 | 15 |
| LEP | 27 | 6 |
| LEP-DOX | 7 | 4 |

### EXAMPLE 5

### Anti-Tumor Efficacy of LEP-DOX on Tumor Bearing Mice

P388/ADR cells (1×105) were injected intravenously (I.V.) on Day 0 in CD2F1 female mice. After 24 hours, mice were randomly divided into different treatment groups (5 mice/group) and vehicle controls or test article formulations were administered I.V. for five consecutive days. Injection volume was based on individual mouse body w eight. Mice were weighed prior to dosing on Days 1-5. Animals were observed for mortality and clinical signs of toxicity. Mean survival time (MST) was determined and the percent increase in lifespan (% ILS) was calculated as follows:
% ILS=[(100×MST of Treatment Group)/(MST of Control Group)]−100% ILS≧25% is considered as a positive response.

Table 8 shows the effect of LEP-DOX on lifespan, suggesting an anti-tumor effect of LEP-DOX.

TABLE 8

| Treatment | % ILS |
|---|---|
| Normal Saline | 0.00 |
| 20 mg/kg LEP | 9.10 |
| 1.0 mg/kg DOX | 18.20 |
| 20 mg/kg LEP + 1.0 mg/kg DOX | 27.28 |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A composition comprising a physiologically acceptable carrier and two or more agents encapsulated into a liposome.

2. The composition of claim 1, wherein the combination of the two or more agents comprises two or more drugs cytotoxic to tumor cells.

3. The composition of claim 1, wherein the combination of the two or more agents exhibits activity against parasites and insects.

4. The composition of claim 1, wherein the combination of the two or more agents exhibits activity against skin penetrating parasites and insects.

5. The composition of claim 1, wherein the combination of the two or more agents is for application to nails, hair, skin or lips.

6. The composition of claim 1, wherein the combination of the two or more agents is a cosmetic.

7. The composition of claim 1, wherein the combination of the two or more agents comprises one or more nutritional products.

8. The composition of claim 1, wherein the combination of the two or more agents is selected from the group consisting of drugs, nutritional supplements, vitamins, minerals, enzymes, hormones, proteins, and peptides.

9. The composition of claim 1, wherein the combination of the two or more agents comprises at least one or more appetite suppressants.

10. The composition of any of claims 1-9, wherein the liposome comprises cardiolipin.

11. The composition of claim 10, wherein the cardiolipin is natural cardiolipin or synthetic cardiolipin.

12. The composition of claim 10, wherein the cardiolipin comprises short-chain fatty acids.

13. The composition of claim 10, wherein the cardiolipin comprises long-chain fatty acids.

14. The composition of claim 10 wherein the liposome further comprises a phosphatidylcholine, cholesterol and α-tocopherol.

15. The composition of claim 10, wherein the liposome further comprises at least one of the lipids selected from the group of lipids consisting of phosphatidylcholine, phosphatidylethanolamine, phosphatidylserine, phosphatidylglycerol, phosphatidic acid, phosphatidylinositol, sphingomyelin, sterol, tocopherol, fatty acid, dimyristoylphophatidylcholine, dimyristoylphophatidylglycerol, dioleoylphosphatidylglycerol, distearoylphophatidyl choline, dioleoylphosphatidylcholine, dipalmitoylphosphatidylcholine, diarachidonoyl phosphatidylcholine, hyrdrogenated soy phosphatidylcholine, and mixtures thereof.

16. The composition of claim 10, wherein the liposome further comprises a sterol selected from a group consisting of cholesterol, polyethylene glycol derivatives of cholesterol, coprostanol, cholestanol, cholestane, cholesterol hemisuccinate, cholesterol sulfate and mixtures thereof.

17. The composition of claim 1, wherein the liposome bears a negative charge.

18. The composition of claim 1, wherein the liposome bears a positive charge.

19. The composition of claim 1, wherein the liposome is neutral.

20. The composition of claim 1, wherein the liposome comprises multilamellar vesicles.

21. The composition of claim 1, wherein the liposome comprises unilamellar vesicles.

22. The composition of claim 1, wherein at least one of the two or more agents is water-soluble, and at least one of the two or more agents is water-insoluble.

23. The composition of claim 1, wherein at least one of the two or more agents is water-soluble, and at least one of the two or more agents is hydrophobic.

24. The composition of claim 1, wherein each of the two or more agents is water-soluble.

25. The composition of claim 1, wherein each of the two or more agents is water-insoluble or hydrophobic.

26. The composition of claim 1, wherein at least one of the two or more agents is present in the aqueous cavity of the liposome, and at least one of the two or more agents is present in the lipid bilayer of the liposome.

27. The composition of claim 1, wherein each of the two or more agents is present in the aqueous cavity of the liposome.

28. The composition of claim 1, wherein each of the two or more agents is present in the lipid bilayer of the liposome.

29. The composition of claim 1, wherein at least one of the two or more agents is selected from the group consisting of: 17α-hydroxyprogesterone acetate, 17-S-estradiol, 19-norprogesterone, 5-fluorouracil, 5-irinotecan, acetazolamide, acetyl sulfisoxazole, adria, adriamycin, adriamycine, alclofenac, allopurinol, alprenolol, aluminum aspirin, aminocaproic acid, amitriptyline, amlodipine, amphetamine sulfate, amphotericin, amphotericin B, anisindone, herceptin, aspirin, atenolol, atropine sulfate, BCNU, bendroflumethiazide, benzamphetamine hydrochloride, bethanechol chloride, bleomycin, calcitonin, calcium gluconate, SN-38, capecitabine, carboplatin, cephalexin, cephalexin hydrochloride, cerubidine, chlordiazepoxide, chlormadinone acetate, chlormethine, chloropromaide, chlorpromazine, chorionic gonadotropin, cimetidine, cisplatin, clonidine, colchicine, corticotrophin, cortisone acetate, cytarabine, cytoxan, cytoxin, daunomycin, daunorubicin, dexamethasone, betamethasone, diazepam, didanosine (ddl), difuinal, digoxin, dihydroxyphenylalanine, diltiazem, diphenadione erythrityl tetranitrate, diphenidol, docetaxel, doctaxel, doxorubicin (including pegylated doxorubicin), EKI-569, enalapril, enalaprilat captopril, epirubicin, erthroxylaceae, erythromycin, erythroxylaceae, Erythroxylacease, ethinyl estradiol, ethinyl estradiol 3-methyl ether, etintidine, etopside, extramustinephosphate, famotidine, felodipine, fenoprofen, fenufen, ferrous sulfate, flufenamic, fluprofen, flurbiprofen, follicle stimulating hormone, gallopamil, gemcitabine, glucagon, gonadotropin releasing hormone, human growth hormone, methione-human growth hormone, des-phenylalanine human growth hormone, bovine growth hormone, porcine growth hormone, insulin-like growth hormone, haloperidol, heparin, herceptin, histermine dihydrochloride, hydrochlorothiazide, hydrocorticosterone acetate, hydrocortisone, hydroxyurea, ibuprofen, idoxide, ifosfamide, imipramine, indomethacin, indoprofen, insulin, insulin-like growth factor, α-interferon, β-interferon, γ-interferon, interferon α-2a, interferon α-2b, consensus interferon, interleukin-2, irinotecan, irinotecan sulindac, isoflurophate, isopropamide iodide, isoproterenol sulfate, isosorbide dinitrate, ketoprofen, leucovorin, leuprolide, levodopa, LHRH, lidoflazine, lisinolpril, luteinizing hormone, lypressin, mandol, mannomustine, mecamylamine hydrochloride, meclizine hydrochloride, mefenamic, melphalan, methacholine chloride, methamphetamine hydrochloride, methazolamide, methotrexate, methyldopa, methylphenidate hydrochloride, methyltestosterone, milrinone, minoxidil, mioflazine, mitobronitol, mitomycin, mitoxantrone, naproxen, nicardipine, nimodipine, nisoldipine, nitrendipine, nitroglycerin, nizatidine, norethiederone, norethindrone, norethisterone, norethynodrel, norgesterone, norgestrel, oxaliplatin, oxytocin, paclitaxel, pancreas hormone releasing factor, pancreozymin, phenaglycodol, phenformin hydrochloride, phenmetrazine hydrochloride, phenoxybenzamine, pilocarpine hydrochloride, prednisolone, procainamide hydrochloride, prochlorperazine maleate, prochlorperzine edisylate, progesterone, prolactin, proleukin, propranolol, quanbenz, raltitrexed, ramipril, ranitidine, reltitrexed, renin, androgens, estrogens, scopolamine bromide, bovine somatotropin, porcine somatotropin, stavudine (d4T), streptozotocin, sucralfate, sulindac, tamoxifen, taxol, tegafur, tetratolol, theophylline, theophylline cholinate, thiethylperzine maleate, thyroid stimulating hormone, thyrotropic hormone, tiapamil, timolol, tolazamide, tolmetin, topotecan, triamcinolone, tridihexethyl chloride, trifosfamide, uramustine, vasopressin, vinblastine, vincamine, vincristine, vinorelbine, xanthins, and zomepirac, and a vaccine against influenza virus, pneumonia, hepatitis A, hepatitis B, hepatitis C, cholera toxin B-subunit, typhoid, plasmodium falciparum, diphtheria, tetanus, herpes simplex virus, tuberculosis, HIV, bordetela pertusis, measles, mumps, rubella, bacterial toxoids, vaccinia virus, adenovirus, canary virus, bacillus calmette, Guerin, or klebsiella pneumonia.

30. The composition of claim 1, wherein at least one of the two or more agents is selected from the group consisting of: agents for treating Alzheimers or Parkinson's disease, agents for treating Crohn's disease, agents for treating demyelinating diseases including multiple sclerosis, agents for treating rheumatology, analgesics, anastrozole, anesthetics, anoretics, anthracyclines, antiallergic agents, anti-arrythmic agents, antibiotics, antibodies, anticoagulants, antidepressants, antidiabetic agents, anti-epilepsy agents, antifungal agents, anti-gout agents, antihypertensive agents, antiinflammatory agents, antiinflammatory corticosteroids, anti-malarials, anti-migraine agents, antimuscarinic agents, anti-protozoal agents, antisense oligonucleotides, anti-thyroids, antiulcer agents, antiulcer drugs, anti-ulcer H2 receptor antagonists, antivirals, anxiolytics, agents for treating arthritis, bisphosphonates, bone morphogenic proteins, camptothecins, cardiac inotropic agents, cardiovascular agents, coagulation factors, corticosteroids, cosmetics, cox-2 inhibitors, cyclosporins, cytokines, derivatives of dexamethasone, dihydropyridines, diuretics, dopaminergic agents, fertility inhibitors, fertility promoters, gastrointestinal agents, glycoproteins, growth factors and hormones, derivatives of human grown hormone, hemostatics, histamine receptor antagonists, hypercholesterol agents, hypnotics, hypocalcemic agents, immunosuppressive agents, immunotoxins, agents for treating inflammatory bowel disease, interferons, interleukins, kidney protective agents, LHRH agonists and antagonists, lipid regulating agents, lipoproteins, moisturizers, muscle relaxants, nephrotoxins, neuroleptics, neurotropic agents, nucleoproteins, nucleotides, oligonucleotides, enzymes, hormones, ophthalmic agents, opioid agonists and antagonists, parasympathomimetics, parathyroid and pituitary hormones, polynucleotides, polypeptides, polysaccharides, prostaglandins, protease inhibitors, proteins, agents for treating psoriasis, retinoids, ribozymes, sedatives, sex hormones, somatostatin, somatotropins, steroids, stimulants, sympathomimetics, taxanes, terpenoids, thyroids, vaccines, and vasodilators.

31. The composition of claim 1, wherein at least one of the two or more agents is a polynucleotide.

32. The composition of claim 31, wherein the polynucleotide is a ribozyme, an interfering RNA(RNAi) or an antisense RNA or DNA sequence.

33. The composition of claim 1 or 32, wherein at least one of the two or more agents is an antisense oligonucleotide.

34. The composition of claim 33, wherein the antisense oligonucleotide is antisense to c-raf.

35. The composition of claim 2, wherein the two or more drugs are selected from the group consisting of paclitaxel, mitoxantrone, SN-38, doxorubicin, gemcitabine, vinorelbine, c-raf antisense oligonucleotide(RafAON), carboplatin, irinotecan, raltitrexed, epirubicin, daunorubicin, cisplatin, topotecan, vinblastine, 5-fluorouracil, mitomycin, adriamycin, capecitabine, dotaxel, didanosine (ddl), stavudine (d4T), hydroxyurea, taxol, interleukin-2, histermine dihydrochloride, tamoxifen, herceptin, cytoxan, leucovorin, oxaliplatin, anastrozole, proleukin, sulindac, EKI-569, and erythroxylaceae.

36. The composition of claim 1, wherein the liposome comprises a c-raf antisense oligonucleotide (RafAON) and at least one drug selected from the group consisting ofpaclitaxel, mitoxantrone, SN-38, doxorubicin, gemcitabine, vinorelbine, vinblastine, cisplatin, 5-flurouracil, mitomycin, and adriamycin.

37. The composition of claim 1, wherein the liposome comprises gemcitabine and at least one drug selected from the group consisting of cisplatin, carboplatin, paclitaxel, topotecan, doxorubicin, and vinorelbine.

38. The composition of claim 1, wherein the liposome is conjugated to a targeting agent that directs binding of the liposome to a tumor cell.

39. The composition of claim 38, wherein the targeting agent is a protein.

40. The composition of claim 39, wherein the protein is selected from the group of proteins consisting of antibodies, antibody fragments, peptides, peptide hormones, receptor ligands, and mixtures thereof.

41. The composition of claim 38, wherein the targeting agent isa ! carbohydrate.

42. A method of treating cancer in a mammalian host, comprising administering to the host a composition comprising: (i) a therapeutically effective amount of a liposome comprising a combination of two or more agents wherein the combination of the two or more agents comprises two or more drugs cytotoxic to tumor cells, and (ii) a physiologically acceptable carrier.

43. The method of claim 42, wherein the mammalian host is a human.

44. The method of claim 42, wherein the liposome comprises cardiolipin.

45. The method of claim 44, wherein the cardiolipin is natural cardiolipin or synthetic cardiolipin.

46. The method of claim 44, wherein the cardiolipin comprises short-chain fatty acids.

47. The method of claim 44, wherein the cardiolipin comprises long-chain fatty acids.

48. The method of claim 44 wherein the liposome further comprises a phosphatidylcholine, cholesterol and α-tocopherol.

49. The method of claim 44, wherein the liposome further comprises at least one of the lipids selected from the group of lipids consisting of phosphatidylcholine, phosphatidylethanolamine, phosphatidylserine, phosphatidylglycerol, phosphatidic acid, phosphatidylinositol, sphingomyelin, sterol, tocopherol, fatty acid, dimyristoylphophatidylcholine, dimyristoylphophatidylglycerol, dioleoylphosphatidylglycerol, distearoylphophatidyl choline, dioleoylphosphatidylcholine, dipalmitoylphosphatidylcholine, diarachidonoyl phosphatidylcholine, hyrdrogenated soy phosphatidylcholine, and mixtures thereof.

50. The method of claim 44, wherein the liposome further comprises a sterol selected from a group consisting of cholesterol, polyethylene glycol derivative of cholesterol, coprostanol, cholestanol, cholestane, cholesterol hemisuccinate, cholesterol sulfate and mixtures thereof.

51. The method of claim 42, wherein the liposome bears a negative charge.

52. The method of claim 42, wherein the liposome bears a positive charge.

53. The method of claim 42, wherein the liposome is neutral.

54. The method of claim 42, wherein the liposome comprises multilamellar vesicles.

55. The method of claim 42, wherein the liposome comprises unilamellar vesicles.

56. The method of claim 42, wherein at least one of the two or more agents is water-soluble, and at least one of the two or more drugs is water-insoluble.

57. The method of claim 42, wherein at least one of the two or more agents is water-soluble, and at least one of the two or more agents is hydrophobic.

58. The method of claim 42, wherein each of the two or more agents is water-soluble.

59. The method of claim 42, wherein each of the two or more agents is water-insoluble or hydrophobic.

60. The method of claim 42, wherein at least one of the two or more agents is present in the aqueous cavity of the liposome, and at least one of the two or more agents is present in the lipid bilayer of the liposome.

61. The method of claim 42, wherein each of the two or more agents is present in the aqueous cavity of the liposome.

62. The method of claim 42, wherein each of the two or more agents is present in the lipid bilayer of the liposome.

63. The method of claim 42, wherein the two or more drugs are selected from the group consisting of paclitaxel, mitoxantrone, SN-38, doxorubicin, gemcitabine, vinorelbine, c-raf antisense oligonucleotide (RafAON), carboplatin, irinotecan, raltitrexed, epirubicin, daunorubicin, cisplatin, topotecan, vinblastine, 5-fluorouracil, mitomycin, adriamycin, capecitabine, dotaxel, didanosine (ddl), stavudine (d4T), hydroxyurea, taxol, interleukin-2, histermine dihydrochloride, tamoxifen, herceptin, cytoxan, leucovorin, oxaliplatin, anastrozole, proleukin, sulindac, EKI-569, and erythroxylaceae.

64. The method of claim 42, wherein the liposome comprises a c-raf antisense oligonucleotide (RafAON) and at least one drug selected from the group consisting of paclitaxel, mitoxantrone, SN-38, doxorubicin, gemcitabine, vinorelbine, vinblastine, cisplatin, 5-flurouracil, mitomycin, and adriamycin.

65. The method of claim 42, wherein the liposome comprises gemicitabine and at least one drug selected from the group consisting of cisplatin, carboplatin, paclitaxel, topotecan, doxorubicin, and vinorelbine.

66. The method of claim 42, wherein the combination of two or more agents is used to treat lung cancer.

67. The method of claim 66, wherein the agents comprise paclitaxel and carboplatin.

68. The method of claim 42, wherein the combination of two or more agents is used to treat non-small cell lung carcinoma.

69. The method of claim 68, wherein the agents are selected from a group consisting of irinotecan, paclitaxel and carboplatin.

70. The method of claim 42, wherein the combination of two or more agents is used to treat urothelial carcinoma.

71. The method of claim 70, wherein the agents comprise gemcitabine and epirubicin.

72. The method of claim 42, wherein the combination of two or more agents is used to treat ovarian carcinoma.

73. The method of claim 72, wherein the agents are selected from a group consisting of gemcitabine, cisplatin, carboplatin, paclitaxel, topotecan, doxorubicin.

74. The method of claim 42, wherein the combination of two or more agents is used to treat melanoma.

75. The method of claim 74, wherein the agents are selected from a group consisting of interleukin-2, histermine dihydrochloride, tamoxifen and cisplatin.

76. The method of claim 42, wherein the combination of two or more agents is used to treat breast cancer.

77. The method of claim 75, wherein the agents are selected from a group consisting of herceptin, paclitaxel, adriamycin, cytoxin, anastrozole, tamoxifen and proleukin.

78. The method of claim 42, wherein the combination of two or more agents is used to treat colorectal cancer.

79. The method of claim 78, wherein the agents are selected from a group consisting of 5-flurouricil, leucovorin, oxaliplatin, 5-irinotecan, irinotecan, sulindac and EKI-569.

80. The method of claim 42 wherein the agents further comprise erythroxylacease and vinblastine.

81. The method of claim 42 or 44, wherein the liposome is conjugated to a targeting agent that directs binding of the liposome to a cell of the cancer.

82. The method of claim 81, wherein the targeting agent is a protein.

83. The method of claim 82, wherein the protein is selected from the group of proteins consisting of antibodies, antibody fragments, peptides, peptide hormones, receptor ligands, and mixtures thereof.

84. The method of claim 81, wherein the targeting agent is a carbohydrate.

85. The method of claim 42 or 44, wherein the liposome is administered dermally, orally, intravenously, or intratumorally.

86. A method of preparing liposomes containing a plurality of active agents formulating a liposomal preparation comprising at least one initial active agent and adding at least one additive active agent to said liposome preparation shortly prior to administration.

87. The method of claim 86, wherein the liposomal preparation comprising at least one initial active agent is in the form of a lyophilized cake, and the additive active agent is added to the cake by first dissolving or suspending the additive active agent in a hydrating solution, which is then added to the cake to reconstitute the liposomes.

88. The method of claim 86 or 87, wherein the initial active agent comprises Paclitaxel.

89. The method of claim 88, wherein the additive active agent comprisesMitoxantrone, anthracycline, or doxorubicin.

90. The method of claim 86 or 87, wherein the initial active agent comprises SN-38.

91. The method of claim 90, wherein the additive active agent comprises gemcitabine.

92. The method of claim 86 or 87, wherein the initial agent is an antisense oligonucleotide.

93. The method of claim 86 or 87, wherein the additive agent is an antisense oligonucleotide.

94. The method of claim 92, wherein the antisense oligonucleotide is antisense to c-raf.

95. The method of claim 86 or 87, wherein the initial agent or the additive agent is one or more agents selected from the group consisting of: 17α-hydroxyprogesterone acetate, 17-S-estradiol, 19-norprogesterone, 5-fluorouracil, 5-irinotecan, acetazolamide, acetyl sulfisoxazole, adria, adriamycin, adriamycine, alclofenac, allopurinol, alprenolol, aluminum aspirin, aminocaproic acid, amitriptyline, amlodipine, amphetamine sulfate, amphotericin, amphotericin B, anisindone, herceptin, aspirin, atenolol, atropine sulfate, BCNU, bendroflumethiazide, benzamphetamine hydrochloride, bethanechol chloride, bleomycin, calcitonin, calcium gluconate, SN-38, capecitabine, carboplatin, cephalexin, cephalexin hydrochloride, cerubidine, chlordiazepoxide, chlormadinone acetate, chlormethine, chloropromaide, chlorpromazine, chorionic gonadotropin, cimetidine, cisplatin, clonidine, colchicine, corticotrophin, cortisone acetate, cytarabine, cytoxan, cytoxin, daunomycin, daunorubicin, dexamethasone, betamethasone, diazepam, didanosine (ddl), difuinal, digoxin, dihydroxyphenylalanine, diltiazem, diphenadione erythrityltetranitrate, diphenidol, docetaxel, doctaxel, doxorubicin (including pegylated doxorubicin), EKI-569, enalapril, enalaprilat captopril, epirubicin, erthroxylaceae, erythromycin, erythroxylaceae, Erythroxylacease, ethinyl estradiol, ethinyl estradiol 3-methyl ether, etintidine, etopside, extramustinephosphate, famotidine, felodipine, fenoprofen, fenufen, ferrous sulfate, flufenamic, fluprofen, flurbiprofen, follicle stimulating hormone, gallopamil, gemcitabine, glucagon, gonadotropin releasing hormone, human growth hormone, methione-human growth hormone, des-phenylalanine human growth hormone, bovine growth hormone, porcine growth hormone, insulin-like growth hormone, haloperidol, heparin, herceptin, histermine dihydrochloride, hydrochlorothiazide, hydrocorticosterone acetate, hydrocortisone, hydroxyurea, ibuprofen, idoxide, ifosfamide, imipramine, indomethacin, indoprofen, insulin, insulin-like growth factor, α-interferon, β-interferon, γ-interferon, interferon α-2a, interferon α-2b, consensus interferon, interleukin-2, irinotecan, irinotecan sulindac, isoflurophate, isopropamide iodide, isoproterenol sulfate, isosorbide dinitrate, ketoprofen, leucovorin, leuprolide, levodopa, LHRH, lidoflazine, lisinolpril, luteinizing hormone, lypressin, mandol, mannomustine, mecamylamine hydrochloride, meclizine hydrochloride, mefenamic, melphalan, methacholine chloride, methamphetamine hydrochloride, methazolamide, methotrexate, methyldopa, methylphenidate hydrochloride, methyltestosterone, milrinone, minoxidil, mioflazine, mitobronitol, mitomycin, mitoxantrone, naproxen, nicardipine, nimodipine, nisoldipine, nitrendipine, nitroglycerin, nizatidine, norethiederone, norethindrone, norethisterone, norethynodrel, norgesterone, norgestrel, oxaliplatin, oxytocin, paclitaxel, pancreas hormone releasing factor, pancreozymin, phenaglycodol, phenformin hydrochloride, phenmetrazine hydrochloride, phenoxybenzamine, pilocarpine hydrochloride, prednisolone, procainamide hydrochloride, prochlorperazine maleate, prochlorperzine edisylate, progesterone, prolactin, proleukin, propranolol, quanbenz, raltitrexed, ramipril, ranitidine, reltitrexed, renin, androgens, estrogens, scopolamine bromide, bovine somatotropin, porcine somatotropin, stavudine (d4T), streptozotocin, sucralfate, sulindac, tamoxifen, taxol, tegafur, tetratolol, theophylline, theophylline cholinate, thiethylperzine maleate, thyroid stimulating hormone, thyrotropic hormone, tiapamil, timolol, tolazamide, tolmetin, topotecan, triamcinolone, tridihexethyl chloride, trifosfamide, uramustine, vasopressin, vinblastine, vincamine, vincristine, vinorelbine, xanthins, and zomepirac, and a vaccine against influenza virus, pneumonia, hepatitis A, hepatitis B, hepatitis C, cholera toxin B-subunit, typhoid, plasmodium falciparum, diphtheria, tetanus, herpes simplex virus, tuberculosis, HIV, bordetela pertusis, measles, mumps, rubella, bacterial toxoids, vaccinia virus, adenovirus, canary virus, bacillus calmette, Guerin, or klebsiella pneumonia.

96. The method of claim 86 or 87, wherein the initial agent or the additive agent is one or more agents selected from the group consisting of: agents for treating Alzheimers or Parkinson's disease, agents for treating Crohn's disease, agents for treating demyelinating diseases including multiple sclerosis, agents for treating rheumatology, analgesics, anastrozole, anesthetics, anoretics, anthracyclines, antiallergic agents, anti-arrythmic agents, antibiotics, antibodies, anticoagulants, antidepressants, antidiabetic agents, anti-epilepsy agents, antifungal agents, anti-gout agents, antihypertensive agents, antiinflammatory agents, antiinflammatory corticosteroids, anti-malarials, anti-migraine agents, antimuscarinic agents, anti-protozoal agents, antisense oligonucleotides, anti-thyroids, antiulcer agents, antiulcer drugs, anti-ulcer H2 receptor antagonists, antivirals, anxiolytics, agents for treating arthritis, bisphosphonates, bone morphogenic proteins, camptothecins, cardiac inotropic agents, cardiovascular agents, coagulation factors, corticosteroids, cosmetics, cox-2 inhibitors, cyclosporins, cytokines, derivatives of dexamethasone, dihydropyridines, diuretics, dopaminergic agents, fertility inhibitors, fertility promoters, gastrointestinal agents, glycoproteins, growth factors and hormones, derivatives of human grown hormone, hemostatics, histamine receptor antagonists, hypercholesterol agents, hypnotics, hypocalcemic agents, immunosuppressive agents, immunotoxins, agents for treating inflammatory bowel disease, interferons, interleukins, kidney protective agents, LHRH agonists and antagonists, lipid regulating agents, lipoproteins, moisturizers, muscle relaxants, nephrotoxins, neuroleptics, neurotropic agents, nucleoproteins, nucleotides, oligonucleotides, enzymes, hormones, ophthalmic agents, opioid agonists and antagonists, parasympathomimetics, parathyroid and pituitary hormones, polynucleotides, polypeptides, polysaccharides, prostaglandins, protease inhibitors, proteins, agents for treating psoriasis, retinoids, ribozymes, sedatives, sex hormones, somatostatin, somatotropins, steroids, stimulants, sympathomimetics, taxanes, terpenoids, thyroids, vaccines, and vasodilators.

97. The method of claim 86 or 87, wherein the initial agent or the additive agent is a polynucleotide.

98. The method of claim 97, wherein the polynucleotide is a ribozyme, an interfering RNA(RNAi) or an antisense RNA or DNA sequence.

99. The method of claim 93 wherein the antisense oligonucleotide is antisense to c-raf.
